(19) United States
(12) Patent Application Publication
(10) Pub. No.: US 2007/0298413 A1
(43) Pub. Date: Dec. 27, 2007

(54) Topoisomerase Modulators Assays

(75) Inventors: Beth Andrews, Waltham, MA (US); Scott Mills, Waltham, MA (US); Maria Uria-Nickelsen, Waltham, MA (US); Wei Yang, Waltham, MA (US); Peter Barth, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 10/551,259

(22) PCT Filed: Mar. 30, 2004
(86) PCT No.: PCT/SE04/00486

§ 371 Date: Aug. 16, 2006

Related U.S. Application Data
(60) Provisional application No. 60/459,187, filed on Mar. 31, 2003.

(51) Int. Cl. C12Q 1/68

(52) U.S. Cl. 435/6

(57) ABSTRACT

The present invention provides methods for screening for compounds that modulate, e.g. inhibit, the activity of topoisomerases such as DNA gyrase, comprising providing cells expressing topoisomerase and containing a promoter sensitive to changes in DNA topology having a reporter gene operatively linked thereto, and measuring the expression of said reporter gene in the presence and in the absence of a test compound. Compounds that modulate the activity of topoisomerase can be identified by virtue of an alteration in reporter gene expression.

### CROSS-REFERENCE TO RELATED APPLICATIONS

This application is related to U.S. Provisional Application 60/459,187, filed Mar. 31, 2000, which is incorporated herein in its entirety.

### FIELD OF THE INVENTION

The present invention relates to screening assays for identifying compounds that modulate the activity of topoisomerase.

### BACKGROUND

DNA topoisomerases all share the property of catalyzing interconversion between different topological forms of DNA. DNA topoisomerases have been isolated from plasmid, viral, prokaryotic, and eukaryotic sources. There are two classes of topoisomerase enzymes (termed type I and type II) that are distinguished by an operational difference; the type I enzymes catalyze DNA interconversion during which the linking number changes in steps of one, while the type II enzymes perform reactions during which the linking number changes in steps of two. Negatively supercoiled DNA is more easily unwound, allowing RNA polymerase to bind more readily to the DNA, hence promoting the transcription of certain genes (Reece & Maxwell, 1991, Crit. Rev. Biochem. Mol. Biol., 26:335-375).

DNA gyrase is a prokaryotic topoisomerase II composed of two separate subunits, encoded by the gyrA and gyrB genes. The GyrA protein functions in the breakage and reunion of DNA, while the GyrB protein has an ATPase activity. All topoisomerases are able to relax negatively supercoiled DNA, but only gyrase can also introduce negative supercoils into DNA.

Bagel et al. (1999, Antimicrobial Agents Chemother., 43:868-875) used the gyrA and topA promoters, in conjunction with the β-lactamase reporter gene, to measure the effect of mutants of gyrase and topoisomerase IV on the degree of DNA supercoiling in E. coli cells. Promoters that respond to the presence of antibiotics have been studied using reporter systems (Fisher et al., 2004, Genome Res., 14:90-98; Shapiro & Baneyx, 2002, Antimicrob Agents Chemother., 46:2490-2497) or expression profiling (Ng et al., 2003, J. Bacteriol., 185:359-370).

### SUMMARY

The present invention provides methods for identifying compounds that modulate topoisomerase activity, comprising providing cells expressing topoisomerase and containing a promoter sensitive to changes in DNA topology having a reporter gene operatively linked thereto, and measuring the expression of said reporter gene in the presence and in the absence of a test compound.

The present invention also provides methods for identifying compounds that modulate DNA gyrase activity, comprising providing cells expressing DNA gyrase and containing a promoter sensitive to changes in DNA topology having a reporter gene operatively linked thereto, and measuring the expression of said reporter gene in the presence and in the absence of a test compound.

### BRIEF DESCRIPTION OF THE DRAWINGS

FIG. 1 shows a graph of the effect of increasing concentrations of the DNA gyrase inhibitor, coumermycin, on β-galactosidase expression in E. coli, using a plasmid containing the dnaA promoter operatively-linked to the lacZ reporter gene.

FIG. 2 shows a diagram of plasmid pBA704 (recF promoter operatively-linked to luxABCDE).

FIG. 3 shows a graph of the effects of increasing concentrations of various compounds on luxABCDE expression in S. aureus, using a plasmid containing the recF promoter operatively-linked to the lux ABCDE operon reporter cassette.

FIG. 4 shows a diagram of the reporter plasmid, pWY428, where the gyrB promoter from H. influenzae was operatively fused to the ZsGreen1 gene (described by Richards et al., 2002, Cytometry, 48:106-112, which is a modified version of the gene described by Matz et al., 1999, Nat. Biotechnol., 17:969-973), and subcloned into vector pVT63 (Trieu & McCarthy, 1990, Gene, 86:99-102).

FIG. 5 shows a graph of the effects of increasing concentrations of ampicillin and novobiocin on ZsGreen1 expression in H. influenzae where the expression of the ZsGreen1 protein is controlled by the gyrB promoter (in pWY428). ZsGreen1 fluorescence is expressed as relative fluorescence units (RFU) per OD₄₉₂ₙₘ of bacterial culture. Concentrations of ampicillin and novobiocin are expressed as fractions of their respective MICs against H. influenzae.

### DETAILED DESCRIPTION

The present invention provides assays for identifying compounds that modulate the activity of topoisomerase. The assays are whole cell reporter assays using cells carrying DNA supercoiling-sensitive promoters that are transcriptionally fused or operatively linked to reporter genes. Modulation of topoisomerase activity results in an alteration of DNA topology that, in turn, causes an alteration in expression of the reporter gene operatively linked to the topology-sensitive promoter. The detection and/or measurement of the expression of the reporter gene can be correlated to the activity of the operatively linked promoter. Hence, compounds that modulate the activity of topoisomerase can be identified by virtue of an alteration in reporter gene expression.

The present invention also provides assays for identifying compounds that inhibit the activity of topoisomerase. The assays are whole cell reporter assays using cells carrying DNA supercoiling-sensitive promoters that are transcriptionally fused or operatively linked to reporter genes. Inhibition of topoisomerase activity results in an alteration of DNA topology that, in turn, causes an alteration in expression of the reporter gene operatively linked to the topology-sensitive promoter. The detection and/or measurement of the expression of the reporter gene can be correlated to the activity of the operatively linked promoter. Hence, compounds that inhibit the activity of topoisomerase can be identified by virtue of an alteration in reporter gene expression.

In some embodiments, the assays of the present invention can be used to identify inhibitors of bacterial topoisomerase and bacterial DNA gyrase for the development of antibacterial agents. The assays of the present invention can be carried out in both Gram-positive and Gram-negative bacterial systems, thereby allowing for the identification of broad spectrum inhibitors.

We have utilized the dnaA promoter operatively-linked to the β-galactosidase reporter gene to develop a cell-based reporter assay in the Gram-negative bacterium Escherichia coli. We have also created a similar construct containing the recF promoter operatively-linked to the lux ABCDE operon reporter cassette for use in the Gram-positive bacterium Staphylococcus aureus. Additionally, we have used the gyrB promoter from Haemophilus influenzae (HI0567) operatively linked to the ZsGreen1 reporter gene to develop a cell-based reporter assay in the Gram-negative bacterium, Haemophilus influenzae. We have used these constructs to show that known gyrase inhibitors can be identified by enhancement of the expression of reporter genes in both Gram-positive and Gram-negative systems.

As used herein, the term “reporter gene expression” refers to any indicators of transcription of the reporter gene. Such indicators include reporter gene transcript products, including mRNA, generated as a result of transcription of the reporter gene, translation products, including all forms of reporter polypeptide or protein and fragments or peptides thereof, generated as a result of translation of reporter gene transcripts, and demonstrable, detectable or otherwise measurable reporter gene product signal and/or activity. The detection and/or measurement and/or quantitation of reporter gene transcript or mRNA, reporter polypeptide, protein, or fragments or peptides thereof, and reporter gene product signal and/or activity is indicative of “reporter gene expression.”

In some embodiments of the present invention, reporter gene expression is detected and/or measured at the transcriptional level (measure and/or detect transcript generated from the reporter gene).

In some embodiments of the present invention, reporter gene expression is detected and/or measured at the translational level (measure and/or detect protein product generated from the reporter gene).

In some embodiments of the present invention, reporter gene expression is detected and/or measured at the activity level (measure and/or detect reporter gene protein product signal and/or activity).

In one aspect, the present invention provides methods for identifying compounds that modulate topoisomerase activity. The methods comprise (a) contacting cells that express a topoisomerase with a test compound, wherein the cells contain a promoter that is sensitive to changes in DNA topology and a reporter gene operatively linked to the promoter, and (b) measuring the reporter gene expression, where an alteration in reporter gene expression in the presence of the compound relative to the absence of the compound is indicative of a compound that modulates topoisomerase activity.

In some embodiments, the assays of the present invention are used to identify compounds that inhibit topoisomerase activity, wherein an alteration in reporter gene expression in the presence of a test compound relative to the absence of the test compound is indicative of a test compound that inhibits topoisomerase activity.

In another aspect, the present invention provides methods for identifying compounds that modulate DNA gyrase activity. The methods comprise (a) contacting cells expressing DNA gyrase with a test compound, wherein said cells contain a promoter sensitive to changes in DNA topology and a reporter gene operatively linked to said promoter; and (b) measuring reporter gene expression, where an alteration in reporter gene expression in the presence of the compound relative to the absence of the compound is indicative of a compound that modulates DNA gyrase activity.

In some embodiments, the assays of the present invention are used to identify compounds that inhibit DNA gyrase activity, wherein an alteration in reporter gene expression in the presence of a test compound relative to the absence of the test compound is indicative of a test compound that inhibits DNA gyrase activity.

Promoter and reporter, gene pairs are selected for use in the assays of the present invention based on a knowledge of: (1) promoters that are transciptionally responsive to compounds, such as novobiocin or ciprofloxicin, that inhibit DNA gyrase in an organism of choice, such as Haemophilus influenzae (Gmuender et al., 2001, Genome Res., 11:28-42) and (2) reporter genes, such as gfp, luxABCDE or lacZ, that function in an organism of choice (Hakkila et al., 2002, Analyt. Biochem, 301:235-242). Such knowledge is within the ordinary skill of the art.

In some embodiments of the present invention, recombinant bacterial strains, carrying a DNA supercoiling-sensitive promoter, such as gyrB, operatively fused to a reporter gene, such as gfp, are grown in the presence of varying concentrations (higher and lower than the MIC) of a control compound, such as novobiocin. As the recombinant strain grows, reporter gene expression is measured and recorded relative to total cellular growth and compared to the strain grown in the absence of the control compound. Depending on the specific promoter and how it responds to cellular DNA topology changes, reporter gene expression will increase or decrease in response to inhibition of DNA gyrase. Validation of the assay with control compounds, as described, will permit one skilled in the art to predict whether test compounds inhibit DNA gyrase based on the behavior of the reporter gene in the assay. Therefore, once the reporter strain is validated with control compounds known to inhibit DNA gyrase, it is used to identify novel compounds as putative DNA gyrase inhibitors. If reporter gene expression changes (in the same direction as the control compound), with increasing concentrations of a potential DNA gyrase inhibitor, the compound would be considered as a putative DNA gyrase inhibitor in whole cells. The compound can then be tested directly for enzymatic inhibition of DNA gyrase. The same scenario is true for other topoisomerases.

As used herein, the terms “modulate” or “modulates” in reference to topoisomerase or DNA gyrase activity includes any measurable alteration, either an inhibition or enhancement, of topoisomerase or DNA gyrase activity. Assays of the present invention utilize reporter genes operably linked to promoters that are sensitive to changes in DNA topology as the basis for detecting topoisomerase or DNA gyrase activity. Any measurable alteration in reporter gene expression can be correlated to a modulation of topoisomerase DNA gyrase activity.

As used herein, the terms “inhibit” or “inhibits” in reference to topoisomerase or DNA gyrase activity includes any measurable diminution of topoisomerase or DNA gyrase activity. Assays of the present invention utilize reporter genes operatively linked to promoters that are sensitive to changes in DNA topology as the basis for detecting topoisomerase DNA gyrase activity. Any measurable alteration in reporter gene expression can be correlated to an inhibition of topoisomerase or DNA gyrase activity.

As used herein, the term “topoisomerase” refers to any topoisomerase from any source, including, but not limited to, topoisomerase I, topoisomerase II and DNA gyrase, topoisomerase III and topoisomerase IV. Topoisomerases have been identified in viruses, plasmids, prokaryotes, and eukaryotes. Any topoisomerase can be assayed using the methods of the present invention. For reviews on topoisomerase, see: Champoux, 2001, Annu. Rev. Biochem., 70:369-413; Wang, 2002, Nat. Rev. Mol. Cell. Biol., 3:430-440.

As used herein, the terms “DNA gyrase” and “gyrase” are used interchangeably to refer to DNA gyrase enzymes.

In some embodiments of the present invention, assays are used to identify compounds that inhibit the activity of DNA gyrase. Any DNA gyrase can be tested in the assays of the present invention, including, but not limited to, DNA gyrase from members of the Enterobacteriaceae family such as Escherichia coli, Salmonella spp, and Shigella spp, DNA gyrase from anaerobes such as Clostridium spp and Bacteroides spp, and DNA gyrase from Haemophilus influenzae, Moraxella catarrhalis, Pseudomonas aeruginosa, Chlamydia spp, Legionella spp, Staphylococcus aureus, Staphylococcus saprophyticus, Streptococcus pneumoniae, Streptococcus pyogenes, Streptococcus mutans, Enterococcus faecalis, Enterococcus faecium, and Mycoplasma spp.

In some embodiments of the present invention, the topoisomerase or DNA gyrase is assayed in the cells in which it is naturally expressed.

In some embodiments of the present invention, the topoisomerase or DNA gyrase is assayed in the cells in which it is not normally or naturally expressed, and the topoisomerase or DNA gyrase is recombinantly expressed in the assay cells. Thus, for example, mammalian topoisomerase may be assayed in bacterial cells, or one species of bacterial DNA gyrase may be assayed in another species of bacterial cell. Nucleic acids encoding topoisomerases have been cloned from many sources, including, but not limited to, bacteria, yeast, mammalian, and viral sources. Cloned mammalian topoisomerases include TopoI: human, mouse, rat, pig, Chinese hamster, dog, and chicken; TopoIIa: human, mouse, rat, pig, Chinese hamster, and bovine; TopoIIb: human, mouse, rat, pig, and Chinese hamster; TopoIIIa: human, mouse, and rat; and TopoIIIb: human and mouse.

Any cell type in which a topoisomerase or DNA gyrase is expressed or can be engineered to be expressed recombinantly, can be used in the assays of the present invention. Such cells include prokaryotic and eukaryotic cell types. Examples of such cells include, but are not limited to, bacterial, archeal, fungal (including Saccharomyces spp, Aspergillus spp and Candida spp), and mammalian (including human).

The terms “promoter sensitive to changes in DNA topology”, “DNA topology-sensitive promoter” and “supercoiling-sensitive promoter” are used interchangeably to refer to polynucleotide sequences that are capable of promoting gene expression and that are responsive to changes in the topology (i.e., linking number) of DNA. Any promoter that is sensitive to changes in DNA topology can be utilized in the methods of the present invention. At least one third of the promoters in E. coli are known to respond to changes in DNA topology in cells (Jovanovich & Lebowitz, 1987, J. Bacteriol., 169:4431-4435). Examples of bacterial promoters that can be used in the assays of the present invention include, but are not limited to, gyrA, gyrB, proU, tppB, ompC, ompF, topA, dnaA, hisD, recF, katE, katG, soda, sodB, tonB, and lacIq mutant. Examples of promoters that can be used in the assays of the present invention are described in the following: Gmuender et al., 2001, supra; O'Byrne et al., 1992, Mol. Microbiol., 6:2467-2476; Bhriain et al., 1989, Mol. Microbiol., 3:933-942; Graeme-Cook et al., 1989, Mol. Microbiol., 3:1287-1294.

The following promoter sequences and functional fragments thereof can be used in the methods of the present invention:

topA promoter:

(SEQ ID NO:1)

5′CGGTCGATGGGTTGTGTCTCTTTGTTCATTATTTACTCCTTAAACAAG
GACATTAGTCTACGCCAGGCATGGCTTGCAGACAAATATACCACGCTGGT
GGCAAGAGCGCCTTACTGGCAACTTTGGATTTTGCATGCTAATAAAGTTG
CGTATCGGATTTTATCAGGTACAGTGTGACGCTTTCGTCAATCTGGCAAT
AGATTTGCTTGACATTCGACCAAAATTCCGTCGTGCTATAGCGCCTGTAG
GCCAAGACCTGTTAACTCAGTCACCTGAATTTTCGTGAACAGAGTCACGA
CAAGGGGTTGATATCCGCAGAGAGCGAGTCCATATCGGTAACTCGTTGCC
AGTGGAAGGTTTATCAACGTGCGACGCATTCCTGGAAGAATCAAATTAGG
TAAGGTGAAT 3′
gyrA promoter:

(SEQ ID NO:2)

5′TGGCACTTCTACTCCGTAATTGGCAAGACAAACGAGTATATCAGGCAT
TGGATGTGAATAAAGCGTATAGGTTTACCTCAAACTGCGCGGCTGTGTTA
TAATTTTGCGACCTTTGAATCCGGGATACAGTAGAGGGATAGCGGTTAG
3′
S. aureus recF promoter:

(SEQ ID NO:3)

5′AAGGTGACGACTCGGTAACGCAATTAATTTTACCAATCAGAACTTACT
AAAAATAAATATAAATAAAGGATGACGTGATTAATTAAAACGTCATCCTT
TATTTTTTGGCAAAAATAATTCTAGATGCGTATGTAAAATAAATTTGACA
GCATTTTAAACAGCAAATAAAAGACGCCAATTAAATTTATGACAAATGTA
TCCAAAATTTAATAAGTGTGCTTATATGCCCTTTAAATTTAAAATTTTAA
TAGTCAATAACAAGTTGAATATTAAAGTTAAACGCCGTTAAATAGCGTTA
AAAAATTGAAAATGACAGTATTGCCAAAAAATAAGAATTAATTATTTATA
TGTAAACGGTTTCTACCTCTATTTTAAATGAAATTTGTGACAAAAAAAGG
TATAATATATTAATGACACACAAAGAAATGGAGTGATTATTTTGGTTCAA
GAAGTTGTAGTAGAAGGAGACATTAATTTAGGTCAATTTCTAAAAACAGA
AGGGATTATTGAATCTGGTGGTCAAGCAAAATGGTTCTTGCAAGACGTTG
AAGTATTAATTAATGGAGTGCGTGAAACACGTCGCGGTAAAAAGTTAGAA
CATCAAGATCGTATAGATATCCCAGAATTACCTGAAGAT 3′
E. coli dnaA promoter:

(SEQ ID NO:4)

5′GATCCTTATTAGATCGATTAAGCCAATTTTTGTCTATGGTCATTAAAT
TTTCCAATATGCGGCGTAAATCGTGCCCGCCTCGCGGCAGGATCGTTTAC
ACTTAGCGAGTTNTGGAAAGTCCTGTGGATAAATCGGGAAAATCTGTGAG
AAACAGAAGATC 3′
Haemophilus influenzae gyrB (HI0567) promoter:

(SEQ ID NO:5)

5′GACCTCGTGGAAATATGCAGCGAGAGGCGCGTAATTCAAGAGGTAATA
ATGTGATAGGCAATGCCTTTGCCTGATGCACTAAAAAATTGGAAAAAATA
ACAAGTTATGGGGCGAAATTATTCGCCCTTTTTTTATCGTTTTCCTTTCC
CGAAAAGCATCGCCAAAACGGCGATTTTTTGCTATAATCTCGCCCAATTT
TTATTTACAAAAGAATGAGATAAATTATG 3′

As used herein, the term “functional fragment thereof” in reference to a promoter sequence means any portion of an identified promoter sequence that retains the function of a promoter that is sensitive to DNA topology and functional in the assays of the present invention.

The promoter sensitive to changes in DNA topology and the reporter gene operatively linked thereto can be provided in a variety of formats, including, but not limited to, on a plasmid, phage, cosmid, other DNA molecules, and provided on the host cell chromosome, either naturally or integrated via recombinant methods known to those of skill in the art.

In some embodiments of the invention, the promoter sensitive to changes in DNA topology and the reporter gene operatively linked thereto are provided on a plasmid or autonomous, self replicating extrachromosomal piece of DNA that is maintained in the cells used in the assay. Any type of plasmid can be used with the assays of the present invention.

In some embodiments low-copy plasmids are used.

In some embodiments medium-copy plasmids are used.

In some embodiments high-copy plasmids are used.

In some embodiments of the invention, the promoter sensitive to changes in DNA topology and the reporter gene operatively linked thereto are provided on a chromosome in the cells used in the assay.

As used herein, the term “reporter gene” refers to any polynucleotide sequence that encodes a polypeptide product whose expression can be detected and/or measured. Reporter genes, their gene products, and methods for the detection or measurement of their expression are well known to those of skill in the art. Any of a wide variety of reporter genes or gene products whose expression can be detected and/or measured can be used with the assays of the present invention, including, but not limited to, lacZ, luxABCDE, luxAB, lucFF, uidA, gfp (green fluorescent protein), RCFPs (Reef Coral Fluorescent Proteins), phoA, kan, and cam.

In some reporter systems the reporter gene product is measured or detected directly by virtue of, for example, chemiluminescent, fluorescent or light producing properties. In some reporter systems the reporter gene product is measured or detected indirectly via the detection or measurement of the activity of the reporter protein on a substrate.

In some embodiments, the reporter gene is a gene encoding a reef coral fluorescent protein (RCFP), such as ZsGreen1, ZsYellow1, AmCyan1, AsRed1 & 2, and DsRed1 & 2. The measurement of the intrinsic fluorescence of RCFPs does not require the addition of a substrate, and continuous fluorescence and absorbance measurements can be taken from the same population of dividing cells. ZsGreen1 has an optimum emission wavelength of 506 nm following excitation at 496 nm (optimum wavelength) (Richards et al., 2002, Cytometry, 48:106-112).

The LacZ reporter protein can be detected by the addition of a reporter substrate: the chromophore signal results from the action of β-galactosidase on a colorless substrate (Miller, 1972, Experiments in Molecular Genetics, p 352-355, Cold Spring Harbor Press, N.Y.). Reporter systems based upon the luxABCDE operon does not require addition of substrate, but rely upon the intrinsic activity of luciferase for detection and can be measured directly when the luxABCDE operon, or a functional equivalent, is expressed (Francis et al., 2000, Infect. Immun., 68:3594-3600; Qazi et al., 2001, Infect. Immun., 69:7074-7082).

For the LacZ reporter system, such substrates as chlorophenolred-β-D-galactopyranoside (CPRG) can be used, which when cleaved by LacZ, undergoes a change in its spectral properties that can be routinely measured. Other examples of reporter systems/substrates include, but are not limited to, the β-lactamase reporter system with a fluorescent/colorimetric β-lactam as a substrate and the phosphatase reporter system with a radio- or immuno-labeled phosphate substrate.

Reporter gene expression is monitored by the method appropriate to the particular reporter system used, including, but not limited to, visual inspection, fluorescence, radiography and others. For example, absorbance is measured for lacZ, luminescence is measured for luxABCDE, and fluorescence is measured for ZsGreen1.

Assay conditions can be routinely optimized by those of skill in the art. Specific parameters for culture medium and growth conditions, reporter gene substrate, adjustments to maximize signal-to-noise ratio and linearity of signal will depend upon the cell type and reporter system used. Such adjustments of parameters are well within the skill of the art.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Screening for Supercoiling-Sensitive Promoters

The T4 terminator (T4 t) and lacZ gene were inserted into a plasmid expression vector pTB244 to create the construct pSB2. In this construct, the transcription of the lacZ gene, without a promoter, is prevented by the upstream T4 terminator, such that insertion of a promoter is necessary for lacZ expression. A random E. coli DNA library was constructed by inserting Escherichia coli chromosomal Sau3A fragments into the BamHI site of the screening vector pSB2, upstream of the promoter-less lacZ gene.

The library was transformed into the lacZ⁻ E. coli host strain MSD1011 (MM294 Δlac=E. coli K12 hsdR Δlac). Transformants, plated onto X-Gal medium, were found to be white or various shades of blue from light to dark. Blue transformants were screened for the presence of relaxation-stimulated promoters using the microtiter plate β-galactosidase assay.

The β-galactosidase assays were carried out in 96-well, flat bottom microtiter plates. Blue (lacZ⁺) ampicillin-resistant colonies of all sizes and shades of blue were purified, inoculated into 200 μl media (L-broth containing 50 μg/ml ampicillin) and incubated overnight at 37° C. Cultures were then diluted 1:20 into fresh broth and incubated for 1.5 hours at 37° C. with shaking at 230 rpm to mid log phase. At this point half of each culture was transferred to a duplicate microtitre plate to which nalidixic acid had been added to give a final concentration of 200 μg/ml. Both plates were incubated at 37° C. for 2 hours to allow expression of the reporter gene that was then assayed. Before carrying out the β-galactosidase assay, absorbance WAS read automatically on the Molecular Devices Microplate Reader at 595 nm in order to measure the differences in the cell titers between control and treated cultures. 25 μl of β-galactosidase assay emulsion (prepared as follows: 5 mL ONPG (O-nitrophenyl-β-D-galactopyranodside) at 4 mg/ml, 5 mL 1× Z-buffer, 0.3 mL 1% SDS, 0.2 mL chloroform, 0.2 mL ether), was added to each well. The plates were read at 28° C. on a Molecular Devices Microplate Reader at 420 nm for 2 mins at 0.09 sec intervals.

The plasmid pRAS101, containing the gyrA promoter linked to a lacZ gene, was used as positive control (Carty & Menzel, 1989, Proc. Natl. Acad. Sci. USA, 86:8882-8886). pRAS101 has a reported stimulation of 3-4-fold when a culture containing it is treated with a gyrase inhibitor.

More than 1000 transformants were tested in duplicate. Clone 19.4 demonstrated a relaxation stimulated transcription ratio of 6.1. The majority of the tested transformants showed reduced expression after treatment with nalidixic acid. Fewer than 10% of the transformants demonstrated positive stimulation ratios, mostly ranging from about 1.5-2.0-fold, with pRAS101 (the positive control) giving an average of 3.1 fold.

### Example 2

### Screening for DNA Gyrase Inhibitors Using a Plate Lawn Technique

The principle of the screen is based on the observation that if known gyrase inhibitors are spotted onto X-Gal medium containing bacteria that carry a DNA relaxation-stimulated lacZ gene on a plasmid, clear zones of inhibition surrounded by a blue ring are formed after overnight growth. The blue ring presumably forms at the drug concentration where optimum relaxation-stimulation of the reporter gene has occurred before the cells are killed.

The following three clones were used: a) clone pRAS101 containing the gyrA promoter linked to the β-galactosidase gene; b) clone 19.4 (found by method described in section 3.3); and c) clone pSB2.his containing the hisD promoter linked to the β-galactosidase gene.

Exemplary Method

Cultures were grown overnight and diluted 1:1000 in (melted) L-agar cooled to 45° C. containing 50 μg/mL Ap and 60 μg/mL X-Gal. The medium was poured into 15×150 mm petri dishes immediately. After it had solidified, known gyrase inhibitors were applied automatically onto the solid agar surface using a Tomtec Quadra 96 machine as follows: 20 μg nalidixic acid (Nx), 100 μg novobiocin (Novo), 2 μg ciprofloxacin (Cipro), 10 μg coumermycin (Cou), 2 μg cinoxacin (Cinox), 2 μg pefloxacin (Pef), 2 μg fleroxacin (Fle), 2 μg flumequine (Flu) and 2 μg norfloxacin (Nor).

Negative controls were placed onto the surface as follows: 25 μg kanamycin (Km), 20 μg chloramphenicol (Cm), 15 μg tetracycline (Tc), 15 μg streptomycin (Sm), 50 μg rifampicin (Rif), 100 μg trimethoprim (Tri), 250 μg sulfathiazole (Su) and 25 μg polymixin B (Px).

Plates were incubated overnight at 37° C.

In one experiment, four gyrase inhibitors and two control antibiotics were spotted onto a lawn of bacteria. Distinct blue rings developed after overnight growth due to increased expression of the DNA relaxation-stimulated reporter gene. Both control drugs (chloramphenicol and tetracycline) showed little to no stimulation. The three clones (pRAS101, 19.4 and pSB2.his) showed similar stimulation responses.

### Example 3

### E. coli Supercoiling Assay Using the LacZ Reporter

Materials

Vector Construct

The dnaA promoter (SEQ ID NO:4, above) was cloned into the BamHI site of the screening vector pSB2 (described in Example 1 above), upstream of the promoter-less lacZ gene.

Part I: Growth of E. coli Cells

A. 250 mL glass flasks

B. Aerobic incubator (37° C.)

C. P-2, P-10, P-200, P-1000 pipettes

D. Sterile pipette tips

E. LB broth

F. Frozen stock of cells

G. Sterile loops

H. Shakers

Part II: Assay

A. P-2, P-10, P-200, P-1000 pipettes

B. DMSO

C. Sterile pipette tips

D. Luria-Bertaini (LB) broth

E. Compounds at 10× the concentration in wells contained in 10% DMSO

F. Microcide inhibitor (phe-Argβ-naphthylamide, Sigma P-4157)

G. 96-well plates

H. CPRG (2 mg/mL)

I. Z buffer (see below)

J. Plate shaker (30° C.)

K. Plate spectrophotometer

Z Buffer Preparation:

Z buffer, adjusted to pH 7, contains in 1 L:

A. 16.1 g Na₂HPO₄7H₂0

B. 5.5 g NaH₂PO₄4H₂0

C. 0.75 g KCl

D. 0.246 g MgSO₄7H₂0

E. 2.7 mL β-mercaptoethanol

Procedure:

Part I: Growth of E. coli Cells

Day 1

1. Frozen culture was inoculated onto LB.

2. The culture was incubated at 37° C. overnight.

Day 2

1. 25 mL of broth was inoculated in a 250 mL flask.

2. The flask was incubated overnight at 37° C., 150 RPM.

Day 3

1. The overnight culture was diluted 1:100 in LB.

2. The culture was incubated at 37° C. and 150 RPM until OD₆₀₀=0.1.

3. The culture was diluted 1:10 in LB.

4. 45 μL of the 1:10 LB culture (step 3) was pipetted into each well of a 96 well-plate containing 5 μL of two-fold dilutions of the test compound in 10% DMSO. The last well of each row contained only 5 μL of 10% DMSO.

5. The plate was incubated at 30° C. and 200 RPM for 2 hours.

6. 50 μL of CPRG (2 mg/ml) and 100 μL of Z buffer was added into all the wells.

7. The plate was incubated at 30° C. and 100 RPM overnight.

8. The OD₅₇₀ was measured and the OD₅₇₀ accounting for the increase in cell mass was subtracted (this value was obtained from a different row in the plate containing all the same ingredients as the other rows except that water was used instead of CPRG) to arrive at “corrected OD₅₇₀”.

9. The “corrected OD₅₇₀” numbers were used to plot the dose-response curves.

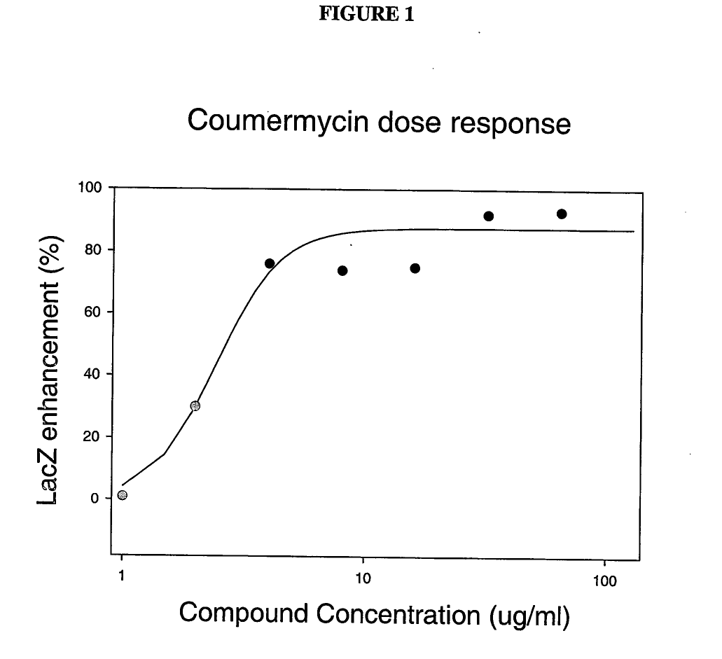

The results with coumermycin are presented in FIG. 1.

### Example 4

### S. aureus Supercoiling Assay Using the luxABCDE Reporter

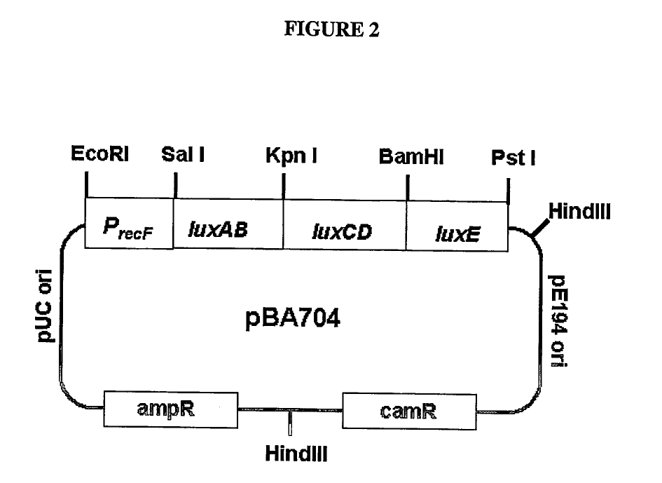

1. The S. aureus strain (RN4220 or ARC516), containing plasmid pBA704 (recF promoter:luxABCDE) (see FIG. 2), was incubated overnight at 37° C., 230 rpm in tryptic soy broth (TSB) with 7 μg/mL chloramphenicol.

2. The overnight culture was diluted in TSB+7 μg/mL chloramphenicol to OD600 0.02, and grown at 37° C., 230 rpm until OD600 reached 0.3.

3. A 96-well plate (Costar solid white flat bottom, cat# 3600) was set up with TSB and test compounds as follows:

A 200 μL aliquot of TSB, containing a test compound (final concentration ˜32×MIC), was placed into the first well.

100 μL of TSB was aliquoted into wells 2 through 12.

Two-fold serial dilutions of the test compound were transferred from well 1 across the plate to well 12.

100 μL was removed from well 12.

50 μL of S. aureus strain (OD600 0.3) was added to each well containing compound.

One additional well was prepared without compound by mixing 100 μL TSB with 50 μL of S. aureus strain (OD600 0.3).

4. A 96-well plate (Costar black, flat bottom, cat# 3711) was set up with TSB and test compounds as follows:

A 200 μL aliquot of TSB, containing a test compound (final concentration ˜32×MIC), was placed into the first well.

100 μL of TSB was aliquoted into wells 2 through 12.

Two-fold serial dilutions of the test compound were transferred from well 1 across the plate to well 12.

100 μL was removed from well 12.

50 μL of S. aureus strain (OD600 0.3) was added to each well containing compound.

One additional well was prepared without compound by mixing 100 μL TSB with 50 μL of S. aureus strain (OD600 0.3).

5. The plates were incubated at 37° C., 230 rpm for up to 3 hours.

6. The luminescence of the cells was measured in the white Costar plate on a Tecan Ultra Evolution using a 200 msec integration time.

7. The OD₄₉₂ of cells in the black Costar plate was measured on a Tecan Ultra Evolution.

8. The relative light units per OD of cells for each well was calculated and the results were plotted as RLU/OD vs compound concentration.

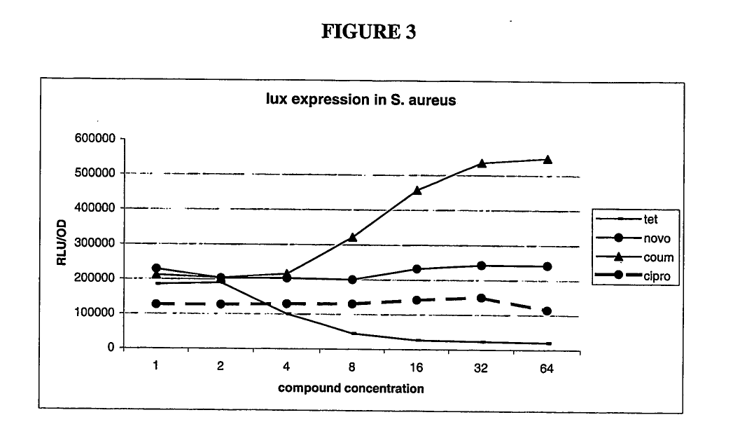

The results are presented in FIG. 3.

### Example 5

### H. influenzae Supercoiling Assay Using a gyrB Promoter-ZsGreen1 Fusion as a Reporter

We have used the gyrB promoter from Haemophilus influenzae gyrB (HI0567) operatively linked to the ZsGreen1 reporter gene to develop a cell-based reporter assay in the Gram-negative bacterium, H. influenzae.

Haemophilus influenzae gyrB (HI0567) promoter:

(SEQ ID NO:5)

5′ GACCTCGTGGAAATATGCAGCGAGAGGCGCGTAATTCAAGAGGTAAT
AATGTGATAGGCAATGCCTTTGCCTGATGCACTAAAAAATTGGAAAAAAT
AACAAGTTATGGGGCGAAATTATTCGCCCTTTTTTTATCGTTTTCCTTTC
CCGAAAAGCATCGCCAAAACGGCGATTTTTTGCTATAATCTCGCCCAATT
TTTATTTACAAAAGAATGAGATAAATTATG 3′

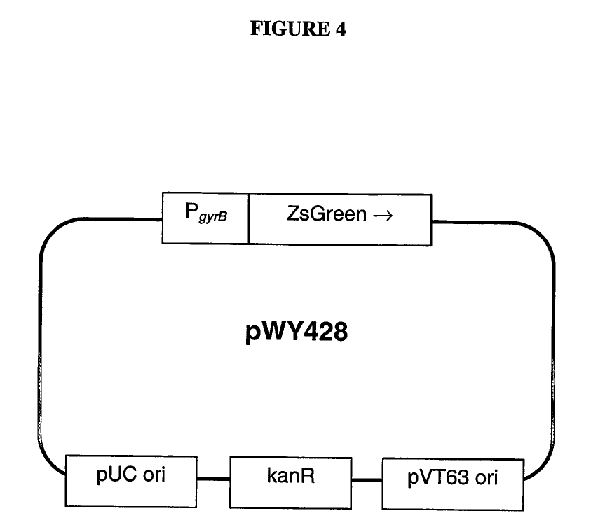

A 5′ C. (shown underlined above) was changed to G to create an AatII (GACGTC) restriction endonuclease site at the 5′ end. At the 3′ end, an AT (shown underlined above) was changed to CA to make a NdeI (CATATG) restriction endonuclease site at the 3′ end. The modified gyrB promoter was cloned into pUC19 using AatII and NdeI sites. An internal NdeI site was removed from ZsGreen1 by site-directed mutagenesis, and then cloned 3′ to the gyrB promoter to make a translation fusion between the promoter and reporter gene. This fusion was then cloned into pVT63 to make pWY428 (FIG. 4).

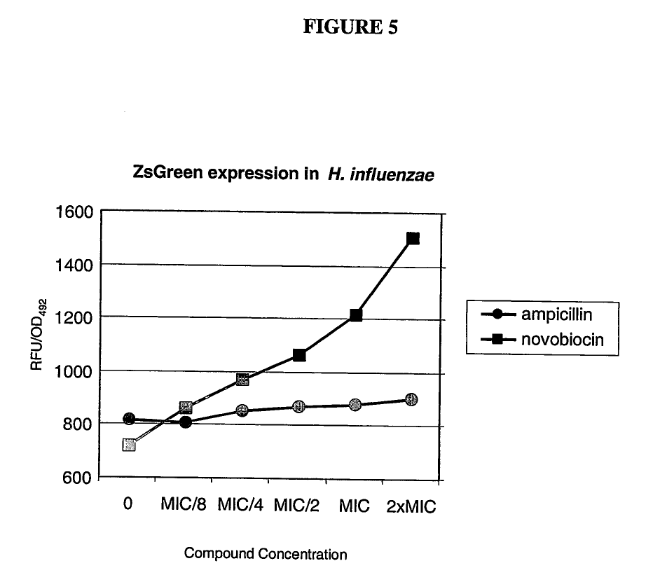

The H. influenzae strain (KW20 or ARC446), containing plasmid pWY428 (gyrB promoter:ZsGreen1) (see FIG. 4), was incubated overnight at 37° C. on supplemented Brain-Heart Infusion (sBHI) agar with 25 μg/ml kanamycin. The overnight culture was suspended to a final OD₆₀₀=0.1 in MIc Minimal Medium and was shaken at 160 rpm at 37° C. until the culture reached an OD₆₀₀=0.4. Media (sBHI and Mic Minimal) were prepaid following standard recipes (Barcak et al., 1991, Methods Enzymol., 204:321-342). 100 μl of culture was added to wells of a 96-well plate (black, clear bottom Costar #3711) that contained 2×, 1×, 0.5×, 0.25×, 0.125×, or 0× of the measured minimum inhibitory concentration (MIC) of ampicillin (MIC=0.125 μg/ml) or novobiocin (MIC=0.125 μg/ml) in an equal volume of DMSO. The plates were shaken at 160 rpm at 37° C. for 3 hours, at which time the OD₄₉₂ was measured with a TECAN Ultra Evolution to record cell density, and fluorescence was measured with a 485 nm (excitation)/535 nm (emission) filter using the TECAN Ultra Evolution. Fluorescence was plotted in FIG. 5 as relative fluorescence units (RFUs) per OD₄₉₂ of bacterial cells at each compound concentration.

The foregoing examples are meant to illustrate the invention and are not to be construed to limit the invention in any way. Those skilled in the art will recognize modifications that are within the spirit and scope of the invention.

1. A method for identifying compounds that modulate topoisomerase activity, said method comprising:
a) providing cells expressing topoisomerase and containing a promoter sensitive to changes in DNA topology having a reporter gene operatively linked thereto;
b) measuring the expression of said reporter gene in the presence and in the absence of a test compound;
c) comparing the expression of said reporter gene in the presence of said compound with the expression in the absence of said compound; and
d) identifying a compound that modulates topoisomerase activity as one that yields an alteration in reporter gene expression in the presence of the compound relative to expression in the absence of the compound.

2. The method of claim 1, wherein modulation of topoisomerase activity is inhibition of topoisomerase activity.

3. The method of claim 1, wherein the topoisomerase is a type II topoisomerase.

4. The method of claim 1, wherein the topoisomerase is a DNA gyrase.

5. The method of claim 1, wherein the topoisomerase is a recombinant topoisomerase.

6. The method of claim 1, wherein the topoisomerase is a prokaryotic, eukaryotic, or viral topoisomerase.

7. The method of claim 1, wherein the promoter is selected from gyrA, gyrB, proU, tppB, ompC, ompF, topA, dnaA, hisD, recF, katE, katG, sodA, sodB, tonB, and laclq mutant, SEQ ID NO:1, SEQ ID NO:2, SEQ ID NO:3, SEQ ID NO:4, SEQ ID NO:5, and any functional fragment thereof.

8. The method of claim 1, wherein the cells are bacterial cells.

9. The method of claim 8, wherein the cells are Gram-positive bacterial cells.

10. The method of claim 8, wherein the cells are Gram-negative bacterial cells.

11. The method of claim 8, wherein the cells are selected from Haemophilus influenzae, Moraxella catarrhalis, Pseudomonas aeruginosa, Escherichia coli, Chlamydia spp, Legionella spp, Staphylococcus aureus, Staphylococcus saprophyticus, Streptococcus pneumoniae, Streptococcus pyogenes, Streptococcus mutans, Enterococcus faecalis, Enterococcus faecium, Mycoplasma spp, Bacteroides spp and Clostridium spp.

12. The method of claim 1, wherein the cells are eukaryotic cells selected from mammalian or fungal cells.

13. The method of claim 12, wherein the cells are human cells, Saccharomyces spp, Aspergillus spp, or Candida spp cells.

14. The method of claim 1, wherein the promoter and reporter gene are provided on a plasmid.

15.-17. (canceled)

19. The method of claim 1, wherein the reporter gene is selected from lacZ, luxABCDE operon, lucFF operon, luxAB operon, uidA, gfp, phoA, kan, cam, and genes encoding reef coral fluorescent proteins.

20.-22. (canceled)

23. A method for identifying compounds that modulate DNA gyrase activity, said method comprising:
a) providing cells expressing DNA gyrase and containing a promoter sensitive to changes in DNA topology having a reporter gene operatively linked thereto;
b) measuring the expression of said reporter gene in the presence and in the absence of a test compound; and
c) identifying a compound that modulates DNA gyrase activity as one that yields an alteration in reporter gene expression in the presence of the compound relative to expression in the absence of the compound.

24. The method of claim 23, wherein the modulation is inhibition of DNA gyrase activity.

25. The method of claim 23, wherein the DNA gyrase is a recombinant DNA gyrase.

26. The method of claim 23, wherein the DNA gyrase is selected from Haemophilus influenzae, Moraxella catarrhalis, Pseudomonas aeruginosa, Escherichia coli, Chlamydia spp, Legionella spp, Staphylococcus aureus, Staphylococcus saprophyticus, Streptococcus pneumoniae, Streptococcus pyogenes, Streptococcus mutans, Enterococcus faecalis, Enterococcus faecium, Mycoplasma spp, Bacteroides spp and Clostridium spp.

27.-45. (canceled)


SEQUENCE LISTING (5 sequences)

SEQ ID NO: 1 - DNA, 408 bp, Escherichia coli
cggtcgatgggttgtgtctctttgttcattatttactccttaaacaaggacattagtctacgccaggcatggcttgcagacaaatataccacgctggtggcaagagcgccttactggcaactttggattttgcatgctaataaagttgcgtatcggattttatcaggtacagtgtgacgctttcgtcaatctggcaatagatttgcttgacattcgaccaaaattccgtcgtgctatagcgcctgtaggccaagacctgttaactcagtcacctgaattttcgtgaacagagtcacgacaaggggttgatatccgcagagagcgagtccatatcggtaactcgttgccagtggaaggtttatcaacgtgcgacgcattcctggaagaatcaaattaggtaaggtgaat

SEQ ID NO: 2 - DNA, 146 bp, Escherichia coli
tggcacttctactccgtaattggcaagacaaacgagtatatcaggcattggatgtgaataaagcgtataggtttacctcaaactgcgcggctgtgttataatttgcgacctttgaatccgggatacagtagagggatagcggttag

SEQ ID NO: 3 - DNA, 637 bp, Staphylococcus aureus
aaggtgacgactcggtaacgcaattaattttaccaatcagaacttactaaaaataaatataaataaaggatgacgtgattaattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatattaatgacacacaaagaaatggagtgattattttggttcaagaagttgtagtagaaggagacattaatttaggtcaatttctaaaaacagaagggattattgaatctggtggtcaagcaaaatggttcttgcaagacgttgaagtattaattaatggagtgcgtgaaacacgtcgcggtaaaaagttagaacatcaagatcgtatagatatcccagaattacctgaagat

SEQ ID NO: 4 - DNA, 160 bp, Escherichia coli
  misc_feature (111)..(111): n is a, c, g, or t
gatccttattagatcgattaagccaatttttgtctatggtcattaaattttccaatatgcggcgtaaatcgtgcccgcctcgcggcaggatcgtttacacttagcgagttntggaaagtcctgtggataaatcgggaaaatctgtgagaaacagaagatc

SEQ ID NO: 5 - DNA, 227 bp, Haemophilus influenzae
gacctcgtggaaatatgcagcgagaggcgcgtaattcaagaggtaataatgtgataggcaatgcctttgcctgatgcactaaaaaattggaaaaaataacaagttatggggcgaaattattcgccctttttttatcgttttcctttcccgaaaagcatcgccaaaacggcgattttttgctataatctcgcccaatttttatttacaaaagaatgagataaattatg